(19) United States
(12) Patent Application Publication
(10) Pub. No.: US 2020/0030540 A1
(43) Pub. Date: Jan. 30, 2020

(54) AUTO-INJECTOR

(71) Applicant: OWEN MUMFORD LTD, Oxfordshire (GB)

(72) Inventors: Matthew John WATTS, Oxfordshire (GB); Robert BRUNS, Oxfordshire (GB); Colin WEBB, Oxfordshire (GB)

(21) Appl. No.: 16/336,917

(22) PCT Filed: Sep. 27, 2017
(86) PCT No.: PCT/GB2017/052886

§ 371 Date: Mar. 27, 2019

(30) Foreign Application Priority Data
Sep. 27, 2016 (GB) 1616393.3
Jul. 14, 2017 (GB) 1711371.3

(51) Int. Cl. A61M 5/315; A61M 5/20; A61M 5/31; A61M 5/46; A61M 5/32

(52) U.S. Cl. CPC A61M 5/3158; A61M 5/2033; A61M 5/3129; A61M 5/46; A61M 2005/2414; A61M 2005/2013; A61M 2005/206; A61M 2005/208; A61M 5/326

(57) ABSTRACT

An auto-injector for receiving a primed syringe includes: a plunger driver for coupling to a plunger head of the syringe when the syringe is fitted within the auto-injector, and configured to drive a plunger into a barrel of the syringe, wherein the plunger driver positioned on the auto-injector such that it is below the plunger head when the syringe is primed and fitted within the auto-injector.

### TECHNICAL FIELD

The invention relates to auto-injectors for use with syringes. The invention may relate to, but need not be limited to, safety auto-injectors and/or auto-injectors for use with safety syringes.

### BACKGROUND

Safety syringes typically include some form of safety mechanism to protect healthcare workers from a hypodermic needle of the syringe after it has been injected into a patient. Exemplary safety syringes may include a shroud or sheath for covering the needle after use of the syringe. Other exemplary syringes may cause the needle to retract within the barrel of the syringe.

Safety syringes may be broadly split into ‘active’ and ‘passive’ safety syringes. Active safety syringes typically require some action by a user of the syringe to engage the safety mechanism. Such action may be taken after removal of the needle from the patient, or may be taken during removal of the needle from the patient. Passive safety syringes typically engage the safety mechanism without any specific action by the user, that is, without any action other than that usually taken to use the syringe.

An auto-injector is a device for receiving a syringe and for driving a syringe plunger of the syringe into a barrel of the syringe without any force being applied by the user.

Typically, an auto-injector includes a plunger driver, such as a spring, that is arranged to provide a force to drive the syringe plunger into the barrel. The plunger driver is typically activated by operation of a button or other release mechanism on the auto-injector. A safety auto-injector may be one which includes a shroud that may be deployed to a position covering a needle of a syringe received within the auto-injector after use of the auto-injector. The shroud of the auto-injector may be deployed under a force applied by a shroud driver.

### SUMMARY

According to an aspect of the invention, there is provided an auto-injector for receiving a primed syringe, the auto-injector comprising: a plunger driver for coupling to a plunger head of the syringe when the syringe is fitted within the auto-injector, and configured to drive a plunger into a barrel of the syringe, wherein the plunger driver positioned on the auto-injector such that it is below the plunger head when the syringe is primed and fitted within the auto-injector.

Optionally, the plunger driver is positioned such that it is below the plunger head after use of the syringe.

Optionally, the auto-injector further comprises a barrel portion configured to receive the barrel of the syringe; and a plunger portion configured to receive the plunger of the syringe when primed, wherein the barrel portion comprises at least part of the plunger driver.

Optionally, the plunger driver is configured to couple to at least one arm coupled to the plunger head and extending towards a needle end of the auto-injector when the syringe is fitted thereto.

Optionally, the plunger driver comprises a biasing means constrained at a first end in relation to the auto-injector and configured to extend towards a needle end of the auto-injector to act upon the at least one arm.

Optionally, the biasing means comprises at least one compression spring.

Optionally, the biasing means forms a channel and is configured to receive the syringe therein, and wherein a second end of the biasing means is configured to couple to the plunger head on insertion of the syringe through the channel.

Optionally, the barrel portion and the plunger portion are separable to expose the channel.

Optionally, the barrel portion comprises a panel slidable between an open position, in which the syringe may be received within the barrel portion, and a closed position.

Optionally, the panel is located on a sidewall of the barrel portion and is configured to slide longitudinally in a direction of a needle end of the auto-injector.

Optionally, the auto-injector further comprises an opening mechanism, coupled to the panel and configured to separate the barrel portion and the plunger portion of the auto-injector on movement of the panel from the closed position to the open position to define a gap for receiving a handle portion of the syringe.

Optionally, the auto-injector further comprises a shroud deployable from a needle-end of the auto-injector to cover at least partially a needle of the syringe after use.

Optionally, the shroud comprises an extension surface configured to couple to the plunger driver, and wherein the plunger driver is further configured to deploy the shroud from the needle end of the barrel portion.

Optionally, the plunger driver is configured such that continued extension thereof couples the second end of the biasing means to the extension surface.

Optionally, the shroud comprises a spring compression surface configured to couple to the plunger driver such that insertion of the shroud into the auto-injector moves the plunger driver into a primed state.

Optionally, the spring compression surface is the extension surface.

Optionally, the auto-injector further comprises a rigid needle shield, RNS, remover coupled to a needle end of the auto-injector and configured for slidable and/or rotatable extension from the auto-injector from first and second positions, the RNS remover configured to couple to an RNS of the syringe when the syringe is received within the auto-injector and to remove the RNS from the needle on movement from the first position to the second position.

Optionally, the RNS remover further comprises an aperture configured to allow the RNS to pass through after removal from the needle.

Optionally, the auto-injector further comprises a locking mechanism coupled to the RNS remover and configured to prevent operation of the auto-injector when the RNS remover is in the first position and to permit operation of the auto-injector when the RNS remover is in the second position.

Optionally, the RNS remover is configured to operate as the shroud when in the second position.

Optionally, the auto-injector further comprises a syringe.

Optionally, the syringe comprises: a plunger having a head; and at least one arm extending longitudinally from the head of the plunger towards a needle end of the syringe and configured to engage with the plunger driver.

### BRIEF DESCRIPTION OF THE DRAWINGS

Exemplary embodiments of the invention are disclosed herein with reference to the accompanying drawings, in which:

FIG. 1 is an isometric view of an auto-injector;

FIG. 2 is a cross section through an auto-injector;

FIG. 3a-1 show an auto-injector at various stages of operation;

FIG. 4 is an isometric view of an auto-injector;

FIGS. 5a-i show an auto-injector at various stages of operation;

FIG. 6a shows an exploded view of an auto-injector;

FIGS. 6b-c show views of a safety syringe fitted within a barrel portion;

FIG. 7 shows a cross section through a contact actuator and depth adjustor;

FIG. 8 shows a cross section through an auto-injector;

FIGS. 9a-b show isometric views of a contact actuator and depth adjustor;

FIGS. 10a-b show isometric views of a depth adjustor and a dial component; and

FIGS. 11a-d show an auto-injector at various stages of operation.

### DETAILED DESCRIPTION

Generally, disclosed herein are auto-injectors for use with a syringe and/or a safety syringe. Exemplary auto-injectors may comprise a plunger driver that is configured to apply a force to a plunger to drive it into a barrel of the syringe and that is located below a head of the plunger of a pre-filled syringe, which may be termed a primed syringe, received within the auto-injector. A syringe is considered to be primed when it contains a drug (or other substance) and has not been used. That is, when the plunger is drawn out of the barrel and there is a substance in the barrel for injection into a subject.

Further, the term “below” the head of the plunger may encompass arrangements where the plunger driver is entirely below the head of the plunger. The plunger driver may be adjacent to or surrounding the barrel of the syringe. Exemplary arrangements allow for a more compact and smaller auto-injector as the plunger driver is not located above the plunger head. Other exemplary arrangements allow the inclusion of other features of the auto-injector above the plunger head because the plunger driver is not located there.

Exemplary plunger drivers are configured to apply the force by coupling to one or more arms extending longitudinally from the head of the plunger longitudinally towards a needle end of the syringe. In an exemplary arrangement, the plunger driver is located beneath an opening of the barrel of the syringe when fitted within the auto-injector. In a specific exemplary arrangement, the plunger driver is located beneath a flange at an opening of the barrel of the syringe when fitted within the auto-injector.

FIG. 1 shows an exemplary auto-injector 100. The auto-injector 100 comprises a barrel portion 102 and a plunger portion 104. The barrel portion 102 is configured to receive a barrel of a syringe and the plunger portion 104 is configured to receive a plunger of the syringe when it is fitted within the auto-injector. Generally, exemplary auto-injectors (and syringes) disclosed herein can be defined as having a needle end 100a and a driver end 100b. These features will be used herein to aid description of the auto-injectors disclosed.

The auto-injector 100 further comprises a rigid needle shield (RNS) remover 106. The RNS remover 106 is configured for slidable extension away from the barrel portion 102 in the direction of the needle end 100a from a first position to a second position. In other arrangements, the RNS remover 106 may be rotated to move it from a first position to a second position. The rotation may result in extension of the RNS remover 106 away from the barrel portion 102. The RNS remover 106 may couple to an RNS of a syringe fitted within the auto-injector 100 such that extension of the RNS remover 106 removes the RNS. An aperture 108 in the RNS remover allows the RNS of the syringe to fall from the auto-injector 100 after removal.

The barrel portion 102 comprises a housing configured to surround a syringe barrel received therein. The barrel portion 102 has a main body that is broadly elliptical in cross section. The main body extends away from the needle end 100b and widens towards an opening configured to receive the barrel of the syringe. The widening may be to accommodate a flange at an opening of the barrel of the syringe or a handle portion that is fitted to the barrel. The handle portion may be configured to receive index and middle fingers of a user when the syringe is used outside of the auto-injector 100. The opening of the barrel portion 102 is configured for removable connection to the plunger portion 104.

The plunger portion 104 comprises a housing configured to surround the plunger of the syringe when fitted within the auto-injector 100. The plunger portion 104 comprises a main body that is broadly elliptical in cross section and may have the same cross sectional dimensions as the main body of the barrel portion 102. The main body of the plunger portion 104 also widens towards an opening to accommodate a flange at an opening of the barrel or a handle portion that is fitted to the barrel. The opening of the plunger portion 104 is configured for removable connection to the barrel portion 102. The plunger portion 104 may further comprise a button 110 or other activation device for activating the auto-injector 100 when a syringe is fitted therein.

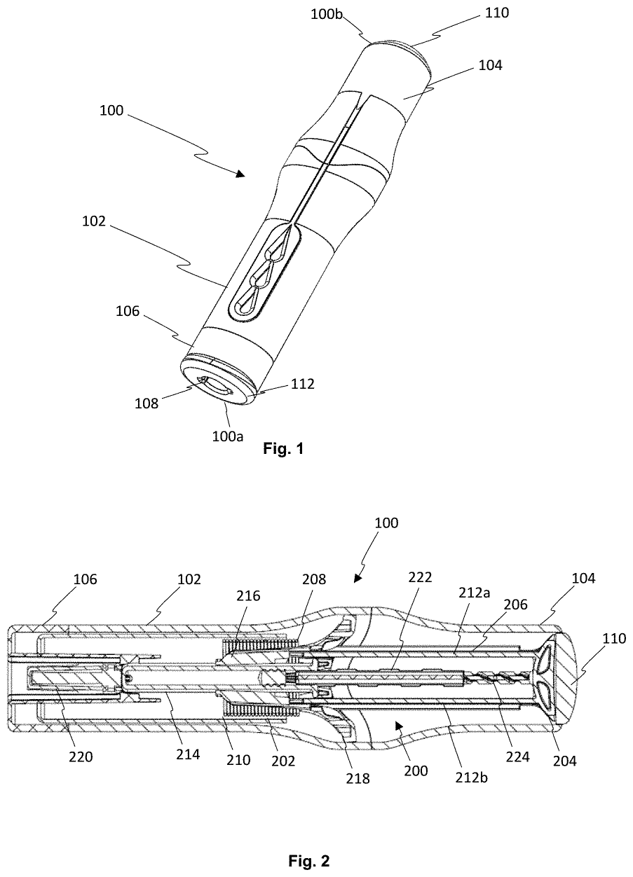

The RNS remover 106 is broadly elliptical in cross section and may have the same cross sectional dimensions as the main body of the barrel portion and/or the plunger portion 104. The RNS remover 106 may comprise a lip 112 around a needle end thereof to provide greater grip for a user wishing to extend the RNS remover 106. FIG. 2 shows a section through the exemplary auto-injector 100 with a syringe 200 fitted therein. The barrel portion 102 comprises a plunger driver 202, which in the exemplary auto-injector 100 is a compression spring but may be another form of driver, such as an extending piston arrangement. In the exemplary arrangement of FIG. 2, the plunger driver 202 is located entirely within the barrel portion 102. The compression spring 202 is located below a head 204 of a plunger 206 of the syringe 200 when fitted to the auto-injector 100. The compression spring 202 is also located such that it is below the head 204 of the plunger 206 when in its primed state and after its use. It is noted that in the above description, the head 204 of the plunger 206 is considered to be the “top” of the syringe and the needle is considered to be the “bottom” of the syringe, but this is to aid description and need not limit the devices disclosed herein. Therefore, if one feature is “below” another feature then it is closer to the needle end.

The compression spring 202 is constrained at a first end 208 relative to the barrel portion 102. In the exemplary arrangement of FIG. 2, the compression spring 202 is connected to the barrel portion 102. The compression spring 202 is a helical spring and defines a channel therethrough. The channel is configured to allow the barrel of the syringe 200 to pass through. A second end 210 of the compression spring is configured to be coupled to at least one arm 212a, 212b of the syringe 200 that is, in turn, coupled to the head 204 of the plunger 206. The second end 210 of the compression spring 202 is closer to the needle end 100a than the first end of the compression spring 202. The arms 212a, 212b extend longitudinally from the plunger head 204 towards the needle end 100a of the auto-injector 100. In the exemplary arrangement of FIG. 2, the arms 212a, 212b extend past the first end 208 compression spring 202 to couple to a lower (second) end 210 of the compression spring 202, such that extension of the compression spring 202 applies a force to the arms 212a, 212b that results in the plunger 206 being driven into a barrel 214 of the syringe 200.

The exemplary syringe 200 shown in FIG. 2 and that is fitted within the auto-injector 100 is a safety syringe. The plunger 206 is a safety plunger coupled to a sheath 216 and comprises the plunger head 204 and the two arms 212a, 212b extending from opposed sides of the plunger head 206. The arms 212a, 212b connect the plunger head 206 to the sheath 216. The arms 212a, 212b are slidable along the outside of the barrel 214 such that the sheath 216 moves along the outside of the barrel 214 on application of a force to the plunger head 204.

A handle portion 218 comprises a main body and flanges extending laterally from the main body. The main body comprises a portion that surrounds the barrel and is fixed thereto. The flanges form a finger grip and, when the safety syringe is used outside of the auto-injector 100, are configured to receive the index finger and middle finger of a user while the thumb applies a force to the plunger head 204 of the safety plunger. The sheath 216 is partially received within the main body of the handle portion 218 when the safety plunger 206 is at the outermost part of its stroke. A portion of the sheath 216 protrudes from the main body of the handle portion 218.

The arms 212a, 212b of the safety plunger 200 pass through the handle portion 218 such that the safety plunger 206 may move along its stroke relative to the handle portion 218 and, therefore, the barrel 214. The sheath 216 is configured to travel along the outside of the barrel 214 with the inward stroke of the safety plunger 206 until the sheath 216 at least partially covers a needle of the safety syringe 200, which in FIG. 2 is covered by an RNS 220. At the innermost point of the stroke of the safety plunger 206, the end of the sheath 216 is beyond the end of the needle, such that the sharp point of the needle is not exposed.

The safety plunger 206 is coupled to a syringe plunger 222 during a first part of its inward stroke and so the syringe plunger 222 is driven into the barrel 214 on movement of the safety plunger 206 along its inward stroke. The safety plunger 206 decouples from the syringe plunger 222 at a point on the inward stroke, which may be when the syringe plunger 222 has expelled all of a medicament or drug from the barrel 214. After decoupling, the safety plunger 206 may move longitudinally relative to the syringe plunger 222 and continued movement of the safety plunger 206 moves the sheath 214 into a position covering the needle. In this sense, decoupling may refer to longitudinal decoupling. There may exist a rotational coupling of the safety plunger 206 and the syringe plunger 222 after longitudinal decoupling.

Decoupling of the safety plunger 206 and the syringe plunger 222 may be by means of relative rotation between the safety plunger 206 and the syringe plunger 222 to disengage one from the other. For example, a decoupling mechanism may comprise a lug on the syringe plunger 222 configured to move between engagement and disengagement with a coupling recess in the safety plunger 206. The movement between engagement and disengagement may be by rotation of the syringe plunger 222 relative to the safety plunger 206.

In some arrangements, the safety syringe 200 may comprise a rate controlling means for controlling a rate of travel of the safety plunger 206 after decoupling. The rate controlling means may comprise a rate limiting member coupled to the safety plunger 206 and configured to engage with the syringe plunger 222. The rate limiting member may comprise a first screw thread 224 and the syringe plunger 222 may comprise a second screw thread (not seen in FIG. 2) that is configured to engage with the first screw thread 224 to rotate the syringe plunger 222 on linear movement of the safety plunger 206 after decoupling.

In such arrangements, the decoupling mechanism may comprise a rotation prevention member configured to prevent rotation of the syringe plunger 222 before decoupling. The rotation prevention member may comprise an aperture through which the syringe plunger 222 passes, wherein the aperture comprises first keying features configured to correspond to second keying features on the syringe plunger 222 such that the syringe plunger 222 is prevented from rotating. The syringe plunger 222 may be configured such that the second keying features disengage from the first keying features to decouple the syringe plunger 222 from the safety plunger 206. For example, the syringe plunger 222 may be configured to pass through the aperture completely to decouple the syringe plunger 222 from the safety plunger 206.

The compression spring 202 may be configured to couple to the sheath 216 of the safety syringe 200 when the safety syringe 200 is inserted into the barrel portion 102. For example, the sheath 216 may comprise a lip or other engagement surface, which may pass the second end 210 of the compression spring 202 on insertion of the syringe 200 through the channel defined by the compression spring 202. For example, the engagement surface may be resiliently deformable such that it is able to deflect inwards as it passes the second end 210 of the compression spring 202 and then to spring back outwards such that the compression spring 202 engages the engagement surface. In this way the second end 210 of the compression spring 202 is directly coupled to the sheath 216 and indirectly coupled to the arms 212a, 212b and plunger head 204. Other arrangements are possible in which the second end 210 of the compression spring 202 is configured to directly couple to the at least one arm 212a, 212b.

The RNS remover 106 comprises spring compression surface configured to engage with the second end 210 of the compression spring 202 such that the compression spring 202 is compressed into a primed (compressed) state when the RNS remover 106 is pushed into the barrel portion 102. When the RNS remover 106 is pushed into the barrel portion 102 after use of the auto-injector 100, the spring compression surface contacts the second end 210 of the compression spring 202 and compresses the compression spring 202 under the force exerted on the RNS remover 106 to push it into the barrel portion 102. The compression spring 202 is compressed into its primed state and is retained there by a latching mechanism. The latching mechanism may be released by depression of the button 110 or other release mechanism to activate the auto-injector 100. In exemplary arrangements, the release mechanism may be activated by depression of the RNS remover 106 against an injection site.

The RNS remover 106 may be configured also to be a shroud that is extendable from the barrel portion 102 to at least partially cover the needle of the syringe 200 after use. In such arrangements, the RNS remover 106 may further comprise an extension surface configured to engage with the second end 210 of the compression spring to extend the RNS remover 106 from the barrel portion 102. In other arrangements, a separate shroud may be used and may comprise an extension surface.

It is noted that in the exemplary arrangement of FIGS. 1 and 2, the plunger driver (or compression spring 202) is located entirely within the barrel portion 102 of the auto-injector 100. The compression spring 202 surrounds the barrel of the syringe when it is fitted within the auto-injector.

The plunger portion 104 comprises a cavity into which the safety plunger 206 and syringe plunger 222 are received when the syringe is primed and is fitted within the auto-injector 100.

The barrel portion 102 and the plunger portion 104 each comprise fixings at the mouths thereof that are configured to cooperate such that the plunger portion 104 may be removably connected to the barrel portion 102.

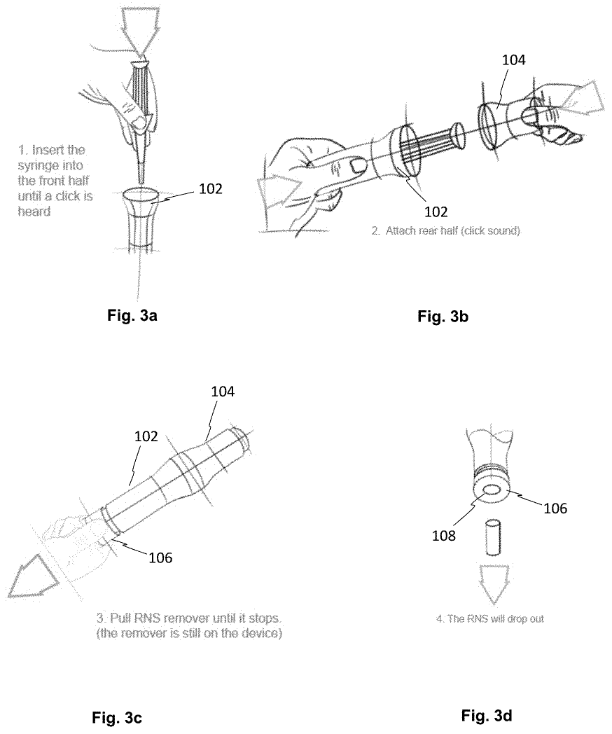

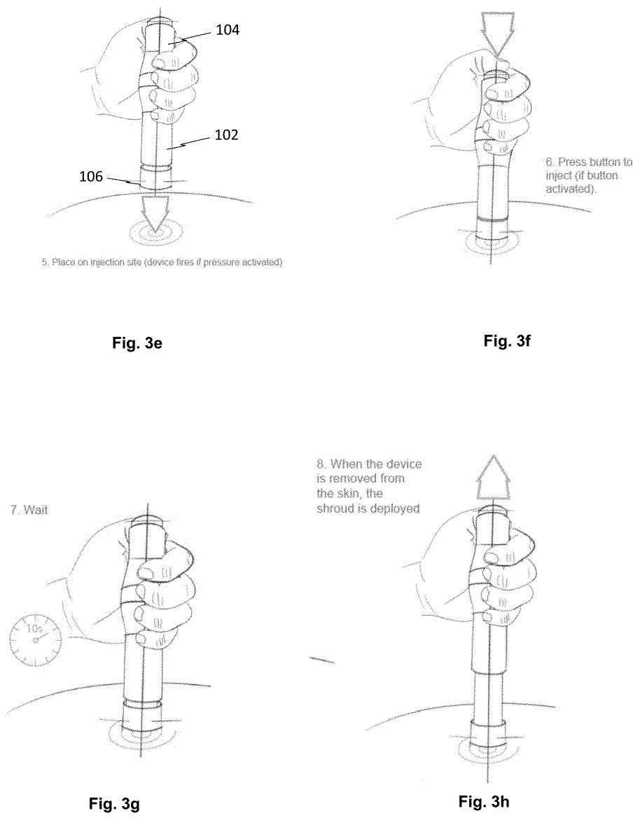

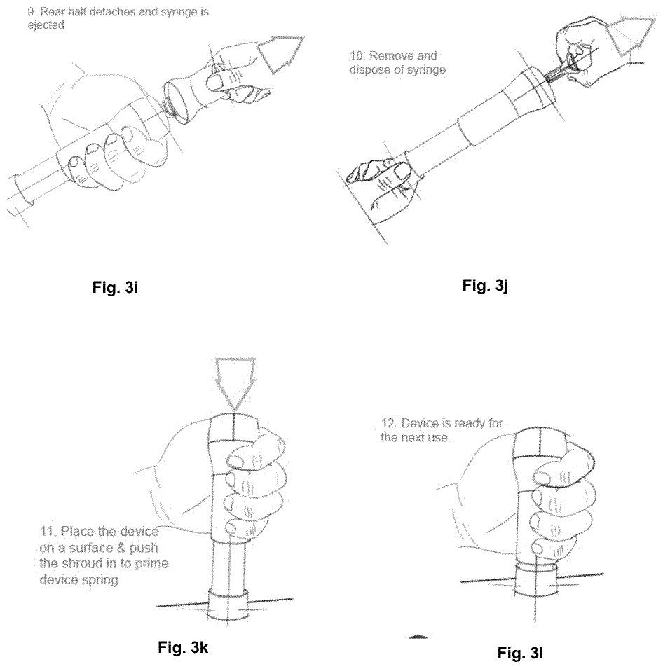

FIGS. 3a-l show the auto-injector 100 of FIGS. 1 and 2 at various stages of use.

In FIG. 3a the barrel portion 102 has been separated from the plunger portion 104 to expose the mouth through which the syringe is inserted into the barrel portion 102 of the auto-injector 100. The compression spring 202 is in its primed state and is latched by the latching mechanism. In the exemplary arrangement of FIGS. 1 and 2, the barrel 214 of the syringe 200 passes through the channel formed by the compression spring 202, which is coupled to the arms 212a, 212b via the sheath 216, as described above. It will be appreciated that in other arrangements a plurality of compression springs (or other plunger drivers) may be used that can be, for example, positioned to be around the periphery of the syringe 200. Also in other arrangements, a syringe may be inserted into the auto-injector 100 when the compression spring 100 (or other plunger driver) is not in a primed (e.g. compressed) state. The compression spring may be primed after insertion of the syringe into the auto-injector 100.

The syringe 200 is inserted into the barrel portion 102 until a click is heard, which signifies that the sheath 216 has connected to or is coupled to the second end 210 of the compression spring 202.

In FIG. 3b the plunger portion 104 is moved over the safety plunger 206 of the syringe 200 such that the safety 206 plunger is received inside the plunger portion 104.

The barrel portion 102 and the plunger portion 104 are connected together.

In FIG. 3c the RNS remover 106 is pulled outwards from the barrel potion 102 until it reaches a mechanical limit. As the RNS remover 106 is coupled to the RNS 220 fitted to the syringe 200, the RNS 220 is removed from the needle and, as shown in FIG. 3d, falls through the aperture 108. The pulling out of the RNS remover 106 activates the release mechanism such that if the RNS remover 106 is depressed against an injection site and/or the button 110 is depressed then the auto-injector 100 will fire.

In FIG. 3e the RNS remover 106 is pressed against the injection site such that the RNS remover 106 is pushed back inside the barrel portion 102. This action may release the latching mechanism such that the compression spring 202 extends, thereby firing the auto-injector 100 and delivering liquid drug to the injection site. In other arrangements, the pushing back of the RNS remover 106 disengages an interlock that otherwise prevents firing of the auto-injector 100. After the interlock is disengaged, depression of the button 110 fires the auto-injector 100

In FIG. 3f the button 110 is depressed by the user, which releases the latching mechanism holding the compression spring 202 in the primed state. The compression spring 202 therefore extends. A first action of the compression spring 202 may be to move the syringe 200 within the auto-injector 100 such that the needle enters the injection site. Extension of the compression spring 202 also pushes the sheath 216 of the syringe 200 towards the needle end 100a. As the sheath 200 is coupled to the plunger head 204 by the arms 212a, 212b, the plunger head 204 is driven down. As the syringe plunger 222 is coupled to the safety plunger 206, the syringe plunger 222 is driven into the barrel 214 to inject a substance contained within the barrel 214 into the injection site.

The auto-injector 100 may be configured such that the compression spring 202 drives the syringe plunger 222 to the bottom of the barrel 214 before it engages with the RNS remover 106. As the RNS remover 106 is in contact with the injection site, the compression spring is prevented from extending any further.

The extension of the compression spring 202 to drive the syringe plunger 222 to the bottom of the barrel 214 takes a period of time, and the user must wait for this, as shown in FIG. 3g.

As shown in FIG. 3h, the user then moves the auto-injector 100 away from the injection site, which allows continued extension of the compression spring 202. The continued extension of the compression spring 202 moves the sheath 216 of the safety syringe 200 over the needle and in the exemplary arrangement of FIGS. 1-3 also moves the RNS remover 106 over the needle. This happens because the compression spring 202 is coupled to the RNS remover 106 either directly or indirectly, for example through the sheath 216 of the safety syringe 200.

After use, the plunger portion 104 of the auto-injector 100 may be disengaged from the barrel portion 102, as shown in FIG. 3i. The safety syringe 200 is then removed from the barrel portion 102, as shown in FIG. 3j. The sheath 216 of the safety syringe 200 is extended over the needle to prevent stick injuries. As shown in FIGS. 3k and 3l, the RNS remover 106 is then pushed into the barrel portion 102 such that the priming surface engages with the compression spring 202 and compresses it into its primed state. The auto-injector 100 may then be stored in two pieces ready for insertion of another syringe at a later time.

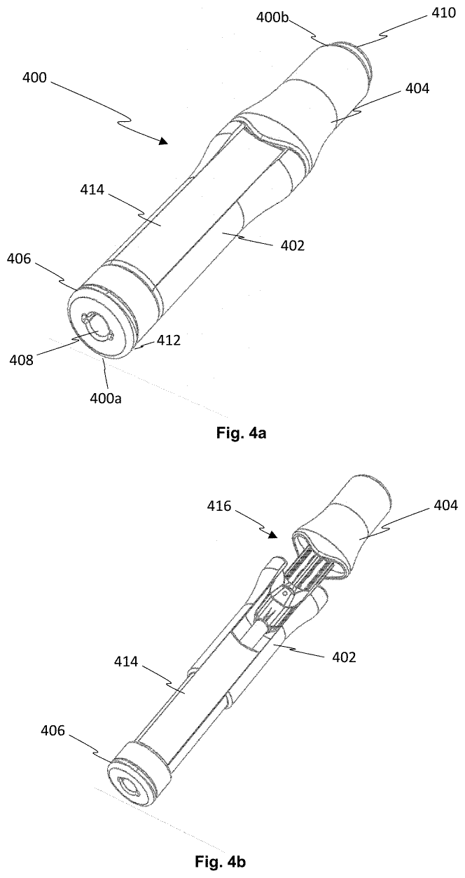

FIGS. 4a and 4b show an exemplary auto-injector 400. Many of the features of the auto-injector 400 are the same or similar to those of the auto-injector 100 discussed above. Accordingly, these features have used the same reference numeral, except prefixed with a “4” instead of a “1”. It is noted that some features of the auto-injector 400 may be used in the auto-injector 100 and vice-versa and such combinations are considered to be disclosed herein.

The auto-injector 400 comprises a barrel portion 402 and a plunger portion 404. The auto-injector 400 has a needle end 400a and a plunger end 400b.

The auto-injector 400 further comprises an RNS remover 406 including an aperture 408 in the RNS remover allows the RNS of the syringe to fall from the auto-injector 400 after removal.

The auto-injector 400 further comprises a panel 414 that is movable between open and closed positions. In the open position, the panel 414 allows insertion of a syringe into the auto-injector 400. In the exemplary auto-injector 400 shown in FIG. 4, the panel is slidable longitudinally towards the needle end 400a of the auto-injector 400 to expose the internal volume of the barrel portion 402.

In the exemplary arrangement of FIGS. 4a and 4b, the RNS remover 406 is configured to connect to the panel 414 such that extension of the RNS remover 406 out of the barrel portion 402 slides the panel 414 longitudinally, as shown in FIG. 4b. The RNS remover 406 may connect to the panel 414 when it is partially extended in order to remove an RNS from a syringe fitted to the auto-injector 400. Further extension of the RNS remover 406 after removal of the RNS opens the panel 414.

When the panel 414 is open, the syringe can be placed into the auto-injector 400 by inserting the plunger into the plunger portion 404 and then laying the barrel of the syringe into the barrel portion 402. The RNS remover 406 may then be pushed back into the barrel portion 402, which closes the panel 414. Continued insertion of the RNS remover 406 after closure of the panel 414 engages the RNS remover 406 with an RNS fitted to the syringe such that later extension of the RNS remover 406 removes the RNS. In exemplary arrangements, inserting the RNS remover 406 into the barrel portion 402 also primes a plunger driver, which may comprise two compression springs on opposed sides of the barrel portion 402. As with the auto-injector 100, the plunger driver is located below the plunger head in both its primed state and after the syringe has been used.

As shown in FIG. 4b, the RNS remover 406 may be coupled to the plunger portion 404 by an opening mechanism. The opening mechanism may be configured to separate the plunger portion 404 from the barrel portion 402 to produce a gap, in which the handle portion of the syringe may be received.

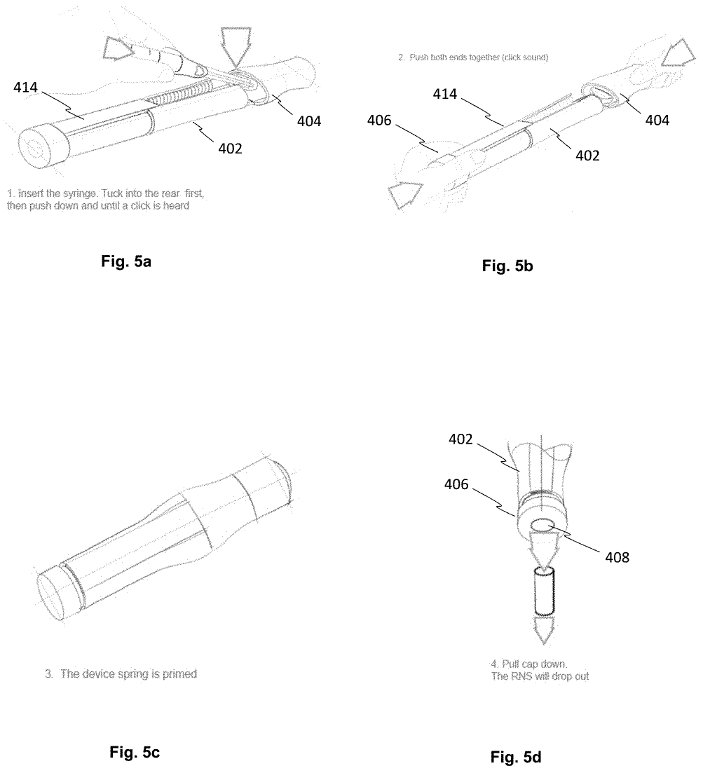

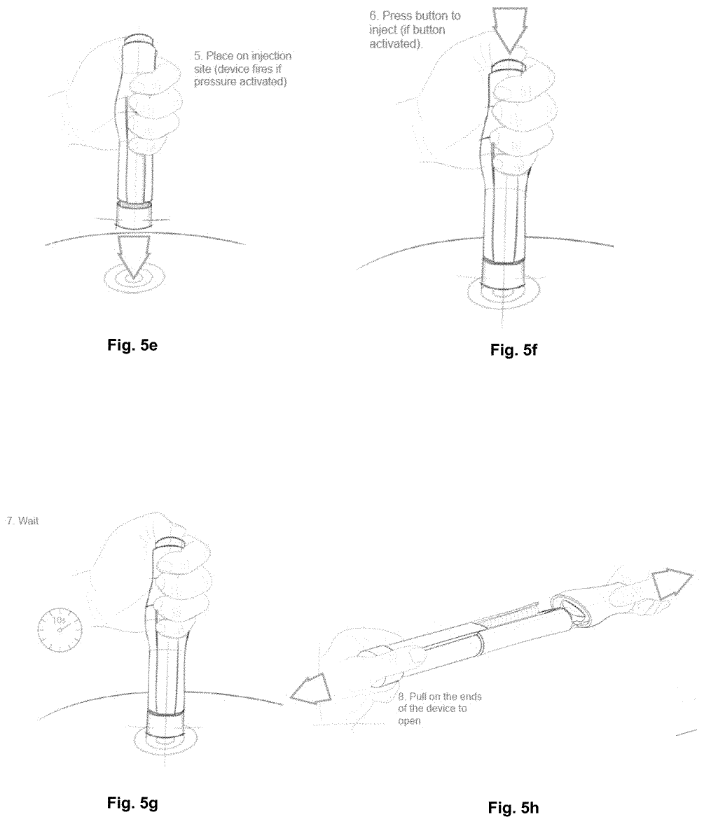

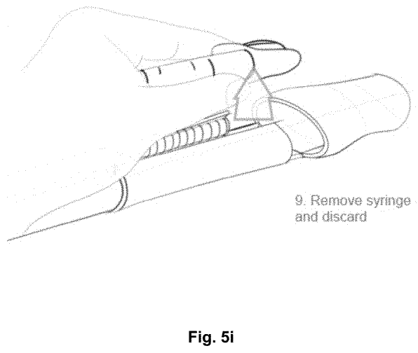

FIGS. 5a-5i show the auto-injector 400 at various stages of use. In FIG. 5a, the panel 414 is shown in its open position such that the internal volume of the barrel portion 402 is exposed and the plunger portion 404 is separated from the barrel portion 402. The plunger of the syringe is inserted into the plunger portion 404 and the barrel of the syringe is laid down into the barrel portion 402. The handle portion is received within the gap created by the separation of the plunger portion 404 and the barrel portion 402. It is noted that in FIGS. 5a-5i the same safety syringe as described above is used with the auto-injector 400.

As shown in FIG. 5b, the plunger portion 404 is pushed towards the barrel portion 402 and the RNS remover 406 is pushed into the barrel portion 402. This action closes the panel 414 and reconnects the plunger portion 404 to the barrel portion 402. The mouth of the plunger portion 404 passes over the handle portion of the syringe to enclose it inside the auto-injector 400. The insertion of the RNS remover 406 into the barrel portion 402 may also prime the plunger driver, which comprises two compression springs on opposed sides of the barrel portion 402. FIG. 5c shows the primed and closed auto-injector 400. The insertion of the RNS remover 406 may also engage the RNS remover 406 with an RNS fitted to the syringe.

As shown in FIG. 5d, before use the RNS remover 406 is pulled outwards from the barrel portion 402 to remove the RNS from the syringe, which then falls from the aperture 408. The pulling out of the RNS remover 406 may activate the release mechanism of the auto-injector 400 such that if the RNS remover 406 is depressed against an injection site and/or the button 410 is depressed then the auto-injector 400 will fire.

In FIG. 5e the RNS remover 406 is pressed against the injection site such that the RNS remover 406 is pushed back inside the barrel portion 402. This action may either fire the auto-injector 400 by actuating the plunger driver or disengage an interlock allowing the plunger driver to be actuated by depression of the button 410.

In FIG. 5f the button 410 is depressed by the user, which releases the latching mechanism holding the two compression springs in their primed states. The compression springs therefore extend pushing the sheath of the syringe towards the needle end 400a. As the sheath is coupled to the plunger head by the arms, the plunger head is driven down. As the syringe plunger is coupled to the safety plunger, the syringe plunger is driven into the barrel.

The extension of the compression springs to drive the syringe plunger to the bottom of the barrel takes a few seconds and the user must wait for this, as shown in FIG. 5g.

After use, the auto-injector 400 is removed from the injection site. The RNS remover 406 is pulled out from the barrel portion 402, which operates the opening mechanism to separate the plunger portion 404 from the barrel portion 402 and slides the panel 414 to the open position. The action of pulling the RNS remover 406 from the barrel portion 402 moves a sheath of the safety syringe to a position covering the needle of the syringe, a captive (safe) position. The syringe can then be removed from the auto-injector, as shown in FIG. 5i. In other arrangements, the RNS remover 406 may be pushed into a position covering the needle by the compression springs. Further, the panel 414 may be opened under a force applied to the RNS remover 406 by the compression springs.

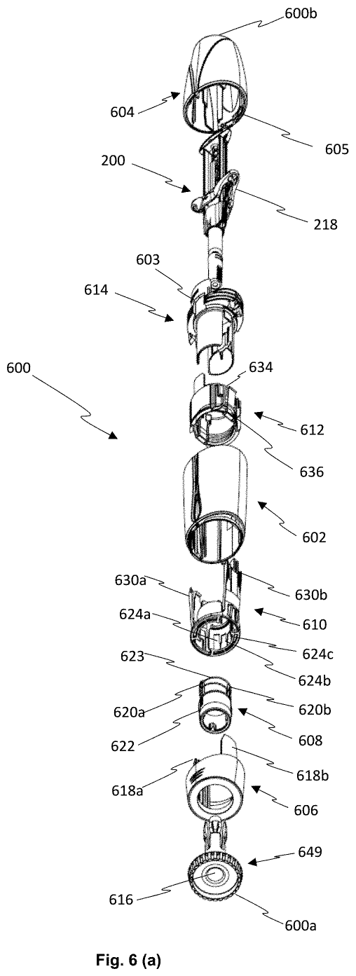

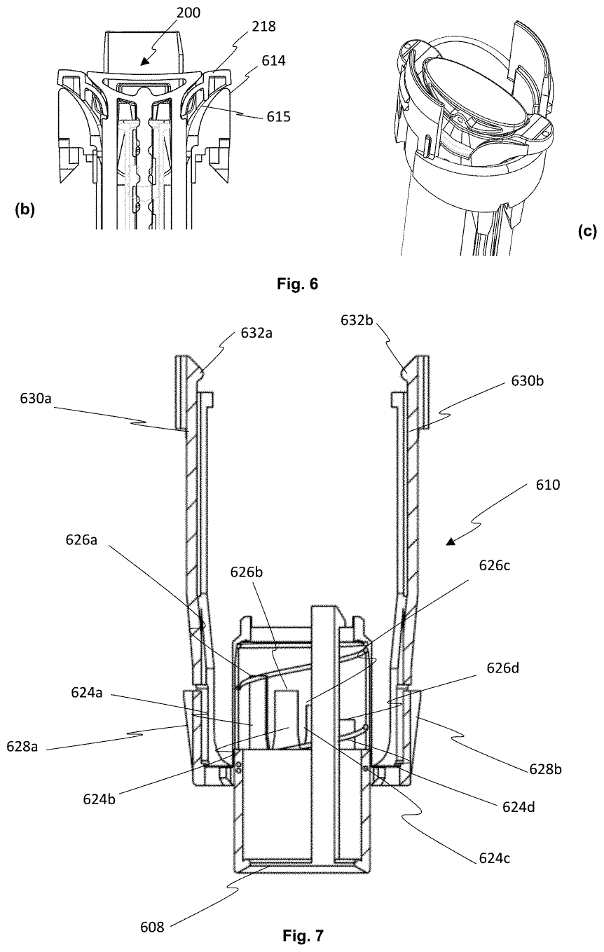

FIG. 6a shows an exploded view of an exemplary auto-injector 600. The auto-injector 600 comprises a barrel portion 602, a plunger portion 604 and a RNS remover 606. The barrel portion 602 houses a depth adjustor 608, a contact actuator 610, a carrier 612 and a body 614. A safety syringe 200 is also shown in FIG. 6a and this may be fitted within the auto-injector 600.

The barrel portion 602 is configured for removable connection with the plunger portion 604.

The plunger portion 604 may be configured for connection to the barrel portion 602 under a linear force applied by a user, and disconnected from the barrel portion 602 under a rotational force. In the exemplary auto-injector 600 comprises discrete threaded sections on both the barrel portion and the plunger portion. The body 614 comprises a barrel thread 603. The body 614 is configured to be fitted within an opening at a plunger end of the barrel portion 602 and comprises at least one guide configured to enter the plunger portion 604 when fitted to the barrel portion 602. The barrel thread is formed on the guide. The plunger portion 604 comprises a corresponding plunger thread 605 positioned at an opening at a needle end thereof.

In the exemplary auto-injector 600, the barrel thread 603 and the plunger thread 605 do not extend 360 degrees around the barrel portion 602 and the plunger portion 604. Rather, the barrel portion 602 and the plunger portion 604 comprise a plurality of discrete threaded sections that are angularly separated from each other about an opening of the barrel portion 602 and the plunger portion 604. In the auto-injector 600, the barrel portion 602 and the plunger portion 604 comprise two thread forms at opposed sides of the openings of the barrel portion 602 and the plunger portion 604, respectively.

In the exemplary auto-injector 600, the linear force applied by the user to connect the barrel portion 602 and the plunger portion 604 may bring the barrel thread 603 and the plunger thread 605 into engagement. This may be by a snap fit or clip fit arrangement which causes the threaded sections to ride over one another under the applied force. Once the barrel thread 603 and the plunger thread 605 are engaged, the plunger portion 604 may be disconnected from the barrel portion 602 by the relative rotation of the barrel portion 602 and the plunger portion 604, which moves the discrete barrel thread 603 out of alignment with the discrete plunger thread 605.

The barrel portion 602 and the plunger portion 604 may be disconnected to allow the insertion of a syringe into the auto-injector 600 and reconnected once the syringe has been inserted. Further, when a syringe 200 is fitted within the auto-injector 600, rotation of the plunger portion 604 with respect to the barrel portion 602 rotates the syringe 200 such that a part of the syringe 200 rides over a cam surface to translate the rotational movement of the syringe to linear movement, which acts to decouple the syringe from the barrel portion 602. Alternatively, rotation of the syringe 200 by rotation of the plunger portion 604 deforms the coupling members retaining the syringe 200 within the barrel portion 602 such that the syringe is decoupled from the barrel portion 602.

In an exemplary arrangement shown in FIGS. 6a and 6b, the barrel portion 602 (or the body 614 within the barrel portion 602 may be shaped to interact with the handle portion 218 of a safety syringe 200 such that a cam surface 615 is formed. The cam surface 615 is configured to translate rotational movement of the safety syringe 200 into linear movement of the safety syringe 200. In the example shown in FIGS. 6b and 6c, the substantially oval opening in the body 614 interacts with the underside of the handle portion 218, which forms a ramped surface, to produce the linear movement. This linear movement may release the syringe 200 from captive coupling with the barrel portion 602, which may extract the syringe 200 from the carrier 612.

The RNS remover 606 is configured for removable connection with the barrel portion 602. The RNS remover 606 may be fully removed (i.e. separable) from the barrel portion 602. The RNS remover 606 may be configured for reattachment to the barrel portion 602 after use of the auto-injector 600 by application of force by a user to insert features of the RNS remover 606 into the barrel portion 602, as explained below.

The RNS remover 606 further comprises prongs 618a, 618b. The prongs 618a-b are configured to be received within the barrel portion 602 when the RNS remover 606 is connected to the barrel portion 602. The prongs 618a, 618b comprise a spring compression surface that is configured to couple to the compression spring 634 when the RNS remover 606 is reconnected to the auto-injector 600 for priming the compression spring 634.

The RNS remover 606 may couple to an RNS of a syringe 200 fitted within the auto-injector 600 such that removal of the RNS remover 606 from the barrel portion 602 removes the RNS. An aperture 616 in the RNS remover allows the RNS of the syringe 200 to fall from the auto-injector 600 after removal of the RNS remover 606. The RNS remover 606 may also comprise a dial component 649 configured to rotate the depth adjustor 608 and operation of which is explained below.

The depth adjustor 608 is configured to partially protrude from the barrel portion 602 of the auto-injector 600 before use thereof. The depth adjustor 608 may be brought into contact with the skin of a user once the RNS remover 606 has been removed. The depth adjustor 608 is configured to be slidably and/or rotatably received within the barrel portion 602 of the auto-injector 600. Slidable movement of the depth adjustor 608 into the barrel portion 602 exposes the needle of the syringe 200 from a needle end of the depth adjustor 608 and therefore allows insertion of the needle into the skin of a patient. In the exemplary auto-injector 600, the depth adjustor is slidably and rotatably received within the contact actuator 610.

The depth adjustor 608 comprises lips 620a-b and a protrusion 622. The lips 620a-b are configured to engage with a surface of the contact actuator 610 to limit extension of the depth adjustor 608 and therefore its protrusion from the barrel portion 602. The depth adjustor may be biased towards a needle end 600a of the auto-injector 600 by a compression spring 623. The protrusions 622 are configured to engage with a mechanical end stop to limit insertion of the depth adjustor into the barrel portion and therefore limit insertion of the needle into the patient.

In the exemplary auto-injector 600 of FIG. 6a, only a single protrusion 622 is visible. However exemplary auto-injectors may comprise a plurality of protrusions 622.

The contact actuator 610 comprises an aperture within which the depth adjustor 608 is received. The depth adjustor 608 may be of a smaller diameter than the diameter of the aperture such that the depth adjustor 608 may be telescopically received by the contact actuator 610. This arrangement allows axial and rotational movement of the depth adjustor 608 with respect to the contact actuator 610.

FIG. 7 shows a section through the depth adjustor 608 and the contact actuator 710 in isolation from the other components of the auto-injector 600. The contact actuator 610 comprises castellation channels 624. Four castellation channels 624a-d are visible in FIG. 7, however exemplary auto-injectors may comprise any number of castellation channels 624a-d.

The castellation channels 624a-d extend longitudinally (with respect to the auto-injector) from the needle end of the contact actuator 610.

The castellation channels 624a-d are different lengths. Each castellation channel 624a-d comprises a mechanical end stop surface 626a-d at a plunger end of the castellation channel 624a-d. Because the castellation channels 624a-d are of different lengths, each mechanical end stop surface 626a-d is located at a different distance longitudinally from the needle end of the contact actuator 610.

The castellation channels 624a-d are configured to receive the protrusion 622 of the depth adjustor 608. The depth adjustor 608 is rotatable within the contact actuator 610 to allow the protrusion 622 to align with the desired castellation channel 624a-d. The castellation channels 624a-d may be configured to allow the protrusion 622 to travel within one of the castellation channels 624a-d. The castellation channels 624a-d may be of substantially the same width as the protrusion 622 such that when the protrusion 622 enters one of the castellation channels 624a-d, rotation of the depth adjustor 608 is prevented and only axial movement of the depth adjustor 608 is possible.

The mechanical end stop surfaces 626a-d are configured to engage with the protrusion 622 of the depth adjustor 608 to set a mechanical limit on the extent of axial movement of the depth adjustor 608 within the contact actuator 610.

The contact actuator 610 further comprises lock-out protrusions 628a-b and arms 630a-b. The arms 630a-b comprise lugs 632a-b configured to engage with the carrier 612 to actuate a plunger driver 634.

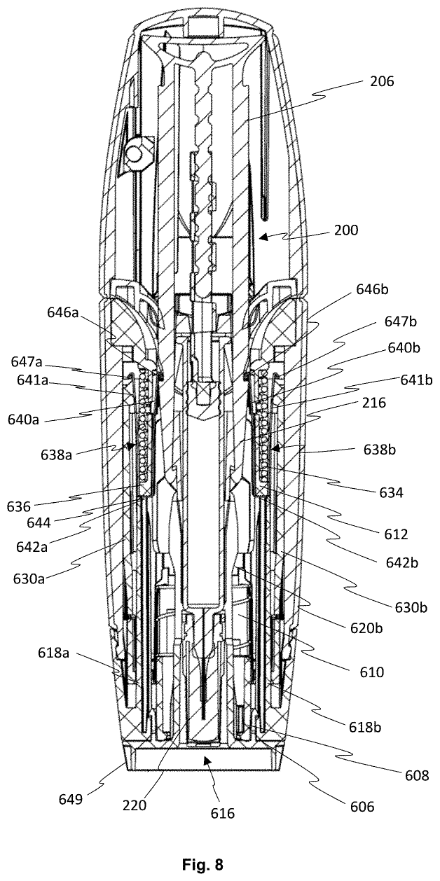

FIG. 8 shows a section through the exemplary auto-injector 600 with a syringe 200 fitted therein. Many of the components of the syringe 200 are the same or similar to those described above and so the same reference numerals are used to refer to features of the syringe 200. A description of these features is not repeated.

The carrier 612 is configured to retain the plunger driver 634 in a primed state. The term “primed state” encompasses a plunger driver that is positioned such that it is capable of applying a biasing force. The plunger driver in the exemplary auto-injector 600 is a compression spring 634. In the exemplary auto-injector 600, the compression spring 634 is received within the carrier 612. The compression spring 634 may be fixed to a biasing surface 636 of the carrier 612. In alternative arrangements, the compression spring 634 may abut the biasing surface 636 without being fixed. The other end of the compression spring 634 may be fixed to or abut a surface of the body 614.

The carrier 612 further comprises clips 638a, 638b located on opposed sides of the carrier 612. Each clip comprises a sheath (or syringe) coupling member 640a, 640b and a locking member 641a, 641b joined by a base 642a, 642b. The sheath coupling members 640a, 640b and the locking members 641a, 641b are resiliently deformable. The sheath coupling members 640a, 640b are angled outwardly (with respect to the longitudinal) towards the body of the barrel portion 602. The locking members 641a, 641b are angled inwardly (with respect to the longitudinal of the auto-injector) towards the centre of the barrel portion 602. As such, each clip 638a, 638b is substantially v-shaped.

The compression spring 634 is received within the clips 638a, 638b such that one end of the compression spring 634 abuts the base 642a, 642b of the clips 638a, 638b and the resiliently deformable members 640a, 640b extend either side of the compression spring 634.

The sheath coupling members 640a, 640b comprise a sheath coupling barb 646a, 646b. The locking members 641a, 641b, comprise a locking barb 647a, 647b. The sheath coupling barbs 646a, 646b are configured to engage with a corresponding recess on the sheath 216 of the syringe 200 when the syringe 200 is inserted within the auto-injector 600. The sheath coupling barbs 646a, 646b couple the sheath 216 to the carrier 612 when the sheath coupling barbs 646a, 646b are engaged in the sheath recess. The locking barbs 647a, 647b are configured to engage with a corresponding recess on the body 614.

Operation of the auto-injector is described below.

Prior to use of the auto-injector 600, the barrel portion 602 is separated from the plunger portion 604. As described above, the barrel portion 602 and plunger portion 604 are separated from each other by rotating the plunger portion 604 relative to the barrel portion 602. This separation exposes an opening in the plunger portion 604 through which the syringe 200 may be inserted.

The syringe 200 is pushed into the barrel portion 602 until a click is heard which signifies that the sheath 216 is coupled to the carrier 612. In the exemplary auto-injector 600, insertion of the syringe 200 into the barrel portion 602 deforms the sheath coupling members 647a, 647b to allow the sheath 216 of the syringe 200 to pass through the carrier 612. As the syringe 200 is pushed into the carrier 612, the sheath coupling barbs 646a, 646b travel along the surface of the sheath 216 until they engage with a sheath recess. The engagement of the sheath coupling barbs 646a, 646b with the sheath recesses couples the carrier 612 to the sheath 216 and therefore to the syringe 200.

The plunger portion 604 is then moved over the safety plunger 206 of the syringe 200 such that the safety plunger 206 is received inside the plunger portion 204. The barrel portion 602 and the plunger portion 604 are connected together. The barrel portion 602 and the plunger portion 604 are connected by the user applying a linear force to snap or clip fit the barrel portion 602 and plunger portion 604 together. This may be done by the barrel thread riding over the plunger thread, as discussed above.

The RNS remover 606 is then disconnected from the barrel portion 602. The RNS remover 606 is pulled outwards from the barrel portion 602 until an inwardly facing lip is disconnected from the corresponding recess on the barrel portion 602. As the RNS remover 606 is coupled to the RNS 220 fitted to the syringe 200, the RNS 220 is removed from the needle and falls through the RNS remover aperture 616.

Disconnecting the RNS remover 606 from the barrel portion 602 exposes the depth adjustor 608.

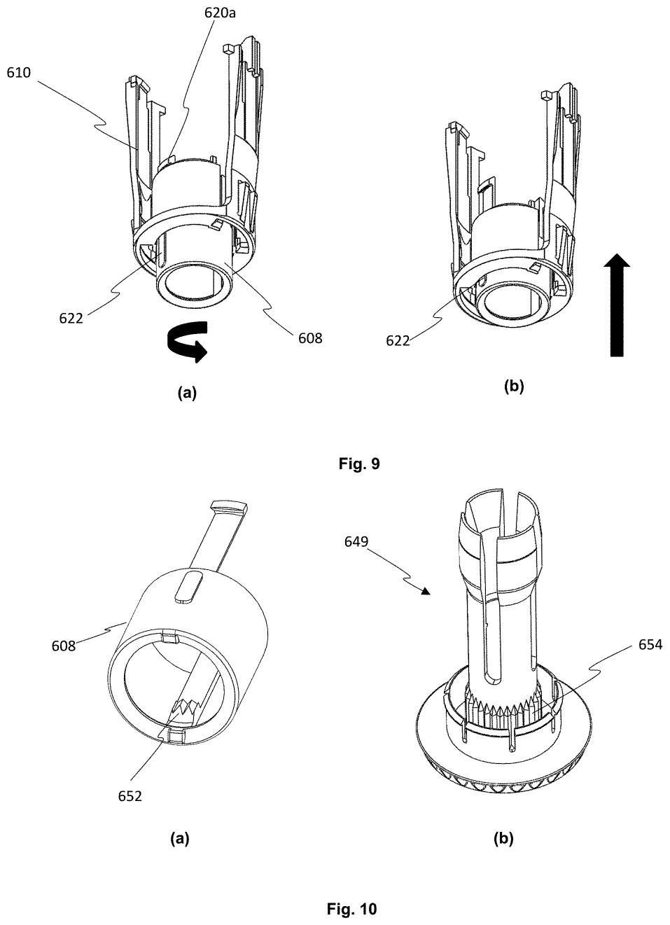

FIGS. 9a-b show isometric views of the depth adjustor 608 and the contact actuator 610 in isolation from the other features of the auto-injector 600.

FIG. 9a shows the depth adjustor 608 once the RNS remover 606 has been disconnected from the barrel portion 602. The depth adjustor 608 is at maximum protrusion from the contact actuator 610 (and therefore the barrel portion 602). At this point the lips 620a, 620b of the depth adjustor 608 are engaged with an upper surface of the contact actuator 610. When the depth adjustor 608 is at a maximum protrusion from the contact actuator 610, the depth adjustor 608 is rotatable with respect to the contact actuator 610. The depth adjustor 608 may be rotated in order to align the protrusion 622 of the depth adjustor 608 with the desired castellation channel 624a-d.

In exemplary auto-injectors 600, the depth adjustor 608 may be rotated before removal of the RNS remover 606 using a dial component 649 which may be received by the RNS remover 606. The dial component 649 may be a separate component to the RNS remover 606 configured for insertion thereof or the dial component 649 may be part of the RNS remover 606. In this arrangement, the depth adjustor may comprise teeth 652 located on an internal surface thereof (see FIG. 10a). The depth adjustor teeth 652 may be configured to engage with corresponding teeth 654 located on the dial component 649 when the RNS remover 606 is connected to the barrel (see FIG. 10b). Rotation of the dial component 649 by the user causes a rotation of the depth adjustor 608 due to the engagement of the depth adjustor teeth 652 with the corresponding teeth 654 on the dial component 649.

By allowing the depth adjustor 608 to be rotated while the RNS remover 606 is still connected to the barrel portion, the depth of needle penetration within the injection site before actuation of the compression 634 is able to be set.

Once the protrusion 622 is aligned with the desired castellation channel 624a-d, axial movement of the depth adjustor 608 within the contact actuator 610 causes the protrusion 622 to enter the castellation channel 624a-d with which it is aligned.

Once the protrusion 622 is aligned with the desired castellation channel 624a-d, the depth adjustor 608 may be pressed against the injection site such that the depth adjustor 608 is pushed inside the contact actuator 610. This causes the protrusion 622 to travel within the selected castellation channel 624a-d. Pushing the depth adjustor 608 inside the contact actuator 610, and therefore inside of the barrel portion 602, exposes and pushes the needle of the syringe 200 into the injection site. The depth adjustor 608 continues to move within the contact actuator 610 and the needle continues to be pushed into the injection site until the protrusion 622 engages with the mechanical end stop surface 626a-d of the castellation channel 624a-d. At this point, the depth adjustor 608 is unable to move within the contact actuator 610 any further and as such, the needle is unable to penetrate into the injection site any further. Selection of a particular castellation channel 624a-d therefore allows the user to customise the depth to which the needle penetrates into an injection site. FIG. 9b shows the relative positions of the depth adjustor 608 and the contact actuator 610 once the protrusion 622 engages with the locking surface 626a-d.

The contact actuator 610 is linearly coupled to the depth adjustor 608. Therefore, once the protrusion 622 engages with the mechanical end stop surface 626a-d of the castellation channel 624a-d, further axial movement of the depth adjustor 608 within the barrel portion 602 causes axial movement of the contact actuator 610 within the barrel portion 602.

As the contact actuator 610 moves rearwards within the barrel portion 602, the lugs 632a, 632b on the arms 630a, 630b of the contact actuator 610 move along the angled surface of the locking members 640a, 640b. This deforms the locking members 640a, 640 and the locking barbs 646a, 646b are disengaged from the recess on the body 614.

The disengagement of the locking barbs 646a, 646b from the recesses on the body 614 releases the carrier 612 holding the compression spring 634 in the primed state.

The compression spring therefore extends. One end of the compression spring is in contact with the biasing surface 636 of the carrier 612. As such, when the compression spring 634 extends, the carrier 612 is pushed towards the bottom of the barrel portion 602.

Because the carrier 612 is coupled to the sheath 216 of the syringe 200, the sheath 216 is also pushed towards the needle end 600a of the auto-injector 600. As the sheath 216 is coupled to the plunger head 204 by the arms 212a, 212b, the plunger head 204 is driven down. As the syringe plunger 222 is coupled to the safety plunger 206, the syringe plunger 222 is driven into the barrel 214 to inject a substance in the barrel 214 into the injection site.

The auto-injector 600 may be configured such that the extension of the compression spring 634 to drive the syringe plunger 222 within the barrel 214 continues until the bottom surface 644 of the carrier 612 is in contact with the injection site. Once the carrier 612 is in contact with the injection site, the compression spring 634 is prevented from extending any further. The auto-injector 600 may be configured such that the carrier 612 makes contact with the injection site once the syringe plunger 222 reaches the bottom of the barrel 214 and all of the substance within the barrel 214 has been expelled.

The extension of the compression spring 634 to drive the syringe plunger 222 to the bottom of the barrel 214 takes a period of time, and the user must wait for this. In exemplary auto-injectors 600, the user must wait for approximately 10 seconds.

The user may then move the auto-injector away from the injection site. This allows for continued extension of the compression spring 634. The continued extension of the compression spring 634 moves the carrier 612 and therefore the sheath 216 (which is coupled to the carrier 612) of the syringe 200 over the needle. The extension of the compression spring 634 continues until the sheath 216 fully covers the needle and the carrier 612 partially protrudes from a needle end opening of the barrel portion 602.

The carrier 612 may comprise an indicator, such as a coloured strip, located on an outer surface of the carrier 612 which is only visible once the extension of the compression spring 634 is completed and the carrier 612 protrudes from the barrel portion 602. This provides a visible indication to the user that the sheath 216 is fully covering the needle and that the syringe 200 is safe for removal from the auto-injector 600.

After use, the plunger portion 604 of the auto-injector 600 may be removed from the barrel portion 602. The barrel portion 602 is rotated relative to the plunger portion 604 to disengage the barrel thread 603 and the plunger thread 605. The rotation of the plunger portion 604, rotates the syringe 200 within the barrel portion 602. This frees the syringe 200 from the carrier 612 by disengaging the sheath coupling barbs 646b, 646c from the recesses located on the sheath 216 of the syringe 200. This may be done using the cam surface 615 or by deformation of the sheath coupling members 640a, 640b, as discussed above.

Once the sheath coupling barbs 646b, 646c are disengaged from the sheath 216, the syringe 200 and the carrier 612 are decoupled and the syringe 200 may be moved independently of the carrier 612.

The syringe 200 may then be removed from the barrel portion 602. The sheath 216 of the syringe 200 is extended over the needle and this prevents stick injuries.

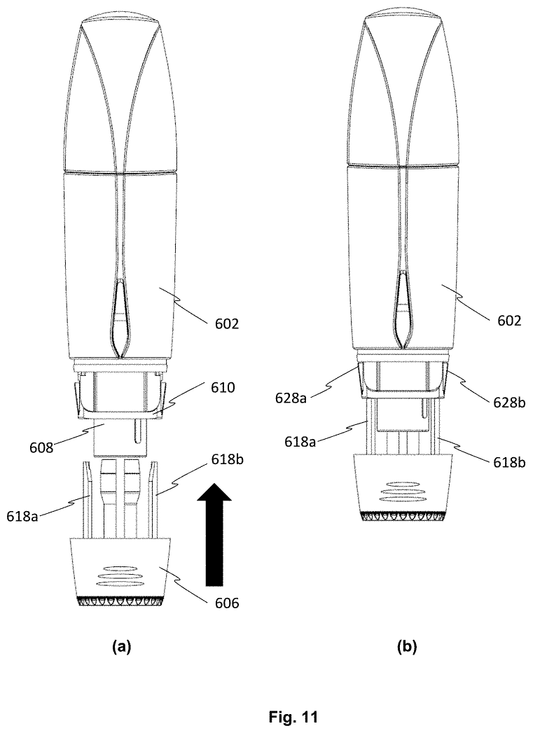

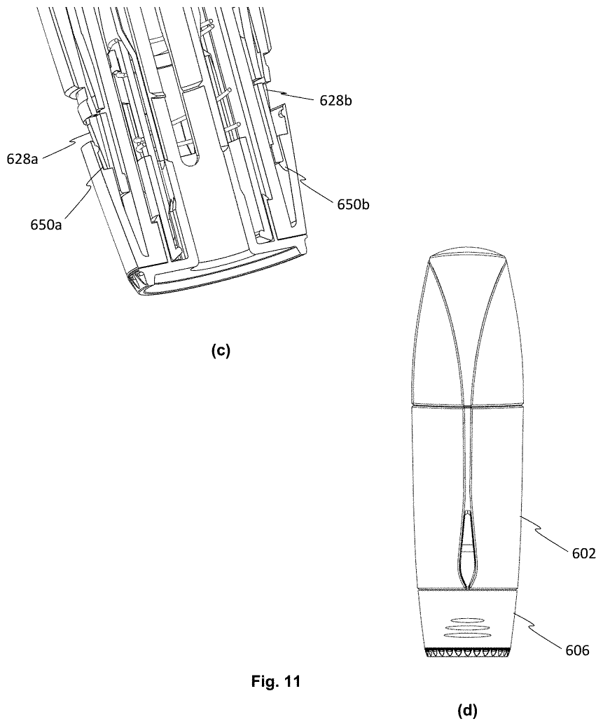

The compression spring 634 may then be primed again for future use of the auto-injector 600. The re-priming operation is described below with reference to FIGS. 11a-d.

In FIG. 11a, the auto-injector 600 has been moved away from the injection site following use of the auto-injector 600 at the injection site and the syringe 200 has been removed from the auto-injector 200. The compression spring 634 is in an extended state, and the contact actuator 610 and carrier 608 protrude from the barrel portion 602. To prime the compression spring 634, the RNS remover 606 is pushed back into the barrel portion 602.

In FIG. 11b the RNS remover 606 is pushed into the barrel portion 602. This causes the prongs 618a, 618b of the RNS remover 606 to engage with the bottom surface 644 of the carrier 612 and push the carrier towards the top of the barrel portion 602. The contact actuator 610 is prevented from being pushed back into the barrel portion 610 along with the carrier 612 by the engagement of the lock-out protrusions 628a-b with the opening of the barrel portion 602.

The lock-out protrusions 628a-b are configured to prevent the contact actuator 610 from being pushed back into the barrel portion 602 until the locking barbs 646a, 646b of the carrier 612 engage with the corresponding recesses on the body 614 of the auto-injector to lock the compression spring 634 into the primed position.

Once the compression spring 634 has been locked into the primed position, further movement of the RNS remover 606 within the barrel portion 602 pushes the lock-out protrusions 628a, 628b inwards.

The lock-out protrusions 628a, 628b may be pushed inwards by ramps 650a, 650b located on an internal surface of the RNS remover 606 (see FIG. 11c). The ramps 650a, 650b may be located at a distance from the bottom of the RNS remover 606 such that they do not interact with the lock-out protrusions until the RNS remover 606 has been pushed within the barrel portion 602 a distance to engage the locking barbs 646a, 646b with the corresponding recesses on the body 614.

Once the lock-out protrusions 628a, 628b have been pushed inwards, further movement of the RNS remover 606 within the barrel portion 602 pushes the contact actuator 610 back into the barrel portion 602 until the RNS remover 606 is reconnected to the barrel portion 602, as shown in FIG. 11d.

It is noted that although the above described methods and apparatus include use of a safety syringe, this need not be the case. Exemplary methods and apparatus can be used with a syringe allowing the plunger driver to be placed below the head of the plunger of the syringe. For example, the methods and apparatus disclosed may comprise a plunger driver configured to couple to the head of the plunger of the syringe from below. This may comprise a direct coupling to the plunger head or an indirect coupling to the plunger head, such as by one or more arms extending from the plunger head towards a needle end of the apparatus.

The skilled person will be able to envisage further embodiments of the invention without departing from the scope of the appended claims.

1-33. (canceled)

34. An auto-injector for receiving a primed safety syringe having a barrel and a plunger and at least one arm extending longitudinally from a head of the plunger and over an end of the barrel to engage a sheath of the safety syringe surrounding and moveable along the outside of said said barrel, the auto-injector comprising:
a plunger driver for coupling to said at least one arm of the syringe when the syringe is fitted within the auto-injector, and configured to thereby drive said plunger into the barrel of the syringe,
wherein the plunger driver is positioned on the auto-injector such that it is below the head of the plunger when the syringe is primed and fitted within the auto-injector.

35. An auto-injector according to claim 34, wherein the plunger driver is positioned such that it is below the head of the plunger after use of the syringe.

36. An auto-injector according to claim 34, further comprising:
a barrel portion configured to receive the barrel of the syringe; and
a plunger portion configured to receive the plunger of the syringe when primed,
wherein the barrel portion comprises at least part of the plunger driver.

37. An auto-injector according to claim 34, wherein the plunger driver comprises a biasing means constrained at a first end in relation to the auto-injector and configured to extend towards a needle end of the auto-injector to act upon the at least one arm.

38. An auto-injector according to claim 37, wherein the biasing means comprises at least one compression spring.

39. An auto-injector according to claim 37, wherein the biasing means forms a channel and is configured to receive the syringe therein, and wherein a second end of the biasing means is configured to couple to the plunger head on insertion of the syringe through the channel.

40. An auto-injector according to claim 36, wherein the barrel portion and the plunger portion are separable to expose the channel.

41. An auto-injector according to claim 40, wherein the barrel portion and the plunger portion each comprise corresponding discrete threaded sections configured such that relative rotation of the barrel portion and the plunger portion misaligns the discrete threaded sections allowing separation of the barrel portion and the plunger portion.

42. An auto-injector according to claim 41, wherein the barrel portion comprises a carrier configured to couple to the sheath when the syringe is fitted within the auto-injector, such that the syringe is retained therein.

43. An auto-injector according to claim 42, wherein the plunger portion is configured to be coupled to the syringe such that rotation of the plunger portion causes rotation of the syringe, the barrel portion further comprising a cam surface configured to extract the syringe from the carrier on rotation of the plunger portion.

44. An auto-injector according to claim 42, wherein the carrier comprises one or more syringe coupling members configured to couple to the sheath when the syringe is fitted within the auto-injector, and wherein the syringe coupling members are deformable on rotation of the plunger portion to decouple the syringe from the carrier.

45. An auto-injector according to claim 36, wherein the barrel portion comprises a panel slidable between an open position, in which the syringe may be received within the barrel portion, and a closed position.

46. An auto-injector according to claim 45, wherein the panel is located on a sidewall of the barrel portion and is configured to slide longitudinally in a direction of a needle end of the auto-injector.

47. An auto-injector according to claim 45, further comprising an opening mechanism, coupled to the panel and configured to separate the barrel portion and the plunger portion of the auto-injector on movement of the panel from the closed position to the open position to define a gap for receiving a handle portion of the syringe.

48. An auto-injector according to claim 34, further comprising a shroud deployable from a needle-end of the auto-injector to cover at least partially a needle of the syringe after use.

49. An auto-injector according to claim 48, wherein the shroud comprises an extension surface configured to couple to the plunger driver, and wherein the plunger driver is further configured to deploy the shroud from the needle end of the barrel portion.

50. An auto-injector according to claim 49, wherein the plunger driver comprises a biasing means constrained at a first end in relation to the auto-injector and configured to extend towards a needle end of the auto-injector to act upon the at least one arm, and wherein the plunger driver is configured such that continued extension thereof couples the second end of the biasing means to the extension surface.

51. An auto-injector according to claim 48, wherein the shroud comprises a spring compression surface configured to couple to the plunger driver such that insertion of the shroud into the auto-injector moves the plunger driver into a primed state.

52. An auto-injector according to claim 51, wherein the shroud comprises an extension surface configured to couple to the plunger driver, and wherein the plunger driver is further configured to deploy the shroud from the needle end of the barrel portion, and the spring compression surface is the extension surface.

53. An auto-injector according to claim 38, wherein the biasing means forms a channel and is configured to receive the syringe therein, and wherein a second end of the biasing means is configured to couple to the plunger head on insertion of the syringe through the channel.
